(19) United States
(12) Patent Application Publication
(10) Pub. No.: US 2021/0059927 A1
(43) Pub. Date: Mar. 4, 2021

(54) COSMETIC COMPOSITION COMPRISING DENDROBIUM CANDIDUM FLOWER EXTRACT

(71) Applicant: LG HOUSEHOLD & HEALTH CARE LTD., Seoul (KR)

(72) Inventors: Byunghyun KIM, Daejeon (KR); Seyeon AHN, Daejeon (KR); So Young LEE, Daejeon (KR); Mu Hyun JIN, Daejeon (KR); Seung-Jin HWANG, Daejeon (KR)

(73) Assignee: LG HOUSEHOLD & HEALTH CARE LTD., Seoul (KR)

(21) Appl. No.: 16/644,101

(22) PCT Filed: Jul. 11, 2018
(86) PCT No.: PCT/KR2018/007832

§ 371 Date: Mar. 3, 2020

(30) Foreign Application Priority Data
Sep. 4, 2017 (KR) 10-2017-0112585

(51) Int. Cl. A61K 8/9794; A61Q 19/02; A61Q 19/08; A23L 33/105

(52) U.S. Cl. CPC A61K 8/9794; A61Q 19/02; A61K 2800/92; A23L 33/105; A61Q 19/08

(57) ABSTRACT

Disclosed is a cosmetic composition including a Dendrobium candidum flower extract, and specifically relates to a cosmetic composition having an excellent skin improvement effect such as wrinkle improvement, skin brightening, skin trouble improvement, skin moisturizing, etc. Also disclosed is a quasi-drug composition and a food composition, which include a Dendrobium candidum flower extract. The cosmetic composition, which includes a Dendrobium candidum flower extract as an active ingredient, is excellent in wrinkle improvement, skin brightening, skin trouble improvement, or skin moisturizing effect. Specifically, the composition is excellent in collagen synthesis promoting effect, melanin production inhibitory effect, anti-inflammatory effect, and moisturizing effect, and has high utility as a cosmetic composition for wrinkle improvement, skin brightening, skin trouble improvement, or skin moisturizing. In addition, the composition, which comprises a Dendrobium candidum flower extract as an active ingredient, can be used as a quasi-drug composition and a food composition.

### TECHNICAL FIELD

The present invention relates to a cosmetic composition comprising a Dendrobium candidum flower extract, and specifically relates to a cosmetic composition having an excellent skin improvement effect such as wrinkle improvement, skin brightening, skin trouble improvement, skin moisturizing, etc. In addition, the present invention relates to a quasi-drug composition and a food composition comprising a Dendrobium candidum flower extract.

### BACKGROUND ART

Collagen is a major substrate protein produced in skin fibroblasts and is present in extracellular stroma, and its important functions are known as mechanical firmness of skin, resistance of connective tissue and binding of tissue, support of cell adhesion, induction of cell division and differentiation (upon the growth of an organism or wound healing), etc. Such collagen is reduced by age and light aging by ultraviolet irradiation, and collagen reduction is promoted by collagenase enzyme activity that degrades collagen. This is known to be closely related to the formation of wrinkles on the skin.

Currently, retinoid, adenosine, animal placental-derived protein, chlorella extracts, etc. are known as skin elasticity enhancement and wrinkle improvement cosmetics. While retinol, which is the best known, is a substance that promotes collagen synthesis and inhibits elastase enzymes, the amount of use is limited due to safety issues such as irritation and redness when applying to skin, and chlorella extracts, etc. are known to have a negligible effect, making it difficult to substantially expect skin elasticity enhancement and wrinkle improvement effects.

Commonly known brightening ingredients include substances that inhibit tyrosinase enzyme activity such as kojic acid, arbutin, etc., hydroquinone, vitamin C (L-ascorbic acid), or derivatives thereof, and various plant extracts. By inhibiting the synthesis of melanin pigments, these can lighten skin tone and achieve skin brightening, and also it is possible to improve skin hyperpigmentation such as blemishes and freckles due to ultraviolet rays, hormones, or heredity. However, when applied on skin, there are limitations on the amount of use due to safety problems such as irritation, redness, etc., or there is a problem that a substantial effect cannot be expected due to a negligible effect.

Cosmetics are products used to protect the skin and to beautify and cleanse the skin, but the composition inevitably includes ingredients that are different from the purpose of protecting the skin for product formation. These ingredients include surfactants, preservatives, fragrances, sunscreens, pigments, as well as other ingredients for imparting other benefits and effects. These ingredients are generally known to cause various problems such as inflammation, rashes, edema, etc. on the skin (Maibach. H. I., Contact Dermatitis, 6. 369-404, 1980). In addition, sebum and sweat, which are released from the body, fatty acids, higher alcohols, protein components, etc. in cosmetics are decomposed into highly toxic substances by skin flora on the skin, which may cause skin inflammation, and it is well known that skin irritation is caused by UV rays from the sun.

Inflammatory responses occur in five phenomena, feeling of redness, pricking feeling, burning hotness, swelling, and change in structure, and these are physiological reactions to protect the living body from harmful environmental conditions, such as invasion by foreign substances such as bacteria and mechanical damage. These inflammatory phenomena lead to a large increase in several polymorphonuclear leukocytes (PMNs) and immune substances, and these increased cells allow for the treatment and defense by releasing various types of proteolytic enzymes, cytokines, etc. which are inflammatory cell products. However, this action can also cause harmful damage to adjacent tissue cells and non-cellular components. Therefore, under proper conditions, after the initial state of inflammation, normal function is restored, but the result is chronic inflammation resulting in much more serious tissue damage, if irritants that irritate inflammation do not disappear or are made continuously.

In general, a cosmetic composition for moisturizing that is used for the skin maintains a certain amount of moisture in the human hair, skin, etc. to make it look soft and lively, and prevents damage such as cracking, dryness, etc. That is, a cosmetic composition for moisturizing the skin is used for the purpose of beautifying the skin or hair and maintaining it to be healthy by supplying a certain amount or more of moisture to the skin or hair, or by maintaining moisture. As the skin is responsible for controlling moisture to be at about 65% to 70%, research on the supply and maintenance of moisture, that is, moisturizing, is active in the field of cosmetics, and recently, the development of cosmetics is active that increase the moisturizing power by maintaining a natural defense function of the skin itself by introducing a biologically active substance obtained from natural products, etc.

For example, Korean Patent No. 2002-0044266 discloses a cosmetic composition for skin protection, which comprises a complex herbal extract. However, herbal extracts alone and their skin protection effect were not disclosed.

### DISCLOSURE

### Technical Problem

Under this background, the present inventors have conducted research on materials that are excellent in wrinkle improvement, skin brightening, skin trouble improvement, and skin moisturizing effect from natural resources that are present in the nature, and found that a Dendrobium candidum flower extract showed superior collagen synthesis promotion, melanin production inhibition, anti-inflammatory effect, and moisturizing effect, compared to extracts from other kinds of candidum and other parts of Dendrobium candidum, and completed a cosmetic composition, a quasi-drug composition, and a food composition utilizing the same.

### Technical Solution

An object of the present invention is to provide a cosmetic composition, comprising a Dendrobium candidum flower extract as an active ingredient.

Specifically, it is to provide a cosmetic composition for wrinkle improvement, comprising a Dendrobium candidum flower extract as an active ingredient.

Specifically, it is to provide a cosmetic composition for skin brightening, comprising a Dendrobium candidum flower extract as an active ingredient.

Specifically, it is to provide a cosmetic composition for skin trouble improvement, comprising a Dendrobium candidum flower extract as an active ingredient.

Specifically, it is to provide a cosmetic composition for skin moisturizing, comprising a Dendrobium candidum flower extract as an active ingredient.

Another object of the present invention is to provide a quasi-drug composition, comprising a Dendrobium candidum flower extract as an active ingredient.

Still another object of the present invention is to provide a food composition, comprising a Dendrobium candidum flower extract as an active ingredient.

### Advantageous Effect

The cosmetic composition of the present invention which comprises a Dendrobium candidum flower extract as an active ingredient, is excellent in wrinkle improvement, skin brightening, skin trouble improvement, or skin moisturizing effect. Specifically, the composition of the present invention is excellent in collagen synthesis promoting effect, melanin production inhibitory effect, anti-inflammatory effect, and moisturizing effect, and has high utility as a cosmetic composition for wrinkle improvement, skin brightening, skin trouble improvement, or skin moisturizing. In addition, the composition of the present invention, which comprises a Dendrobium candidum flower extract as an active ingredient, can be used as a quasi-drug composition and a food composition.

### BEST MODE

As an aspect for achieving the above object, the present invention provides a cosmetic composition comprising a Dendrobium candidum flower extract as an active ingredient.

In the present invention, “Dendrobium candidum” is one of the orchid family (Orchidaceae) plants well known as traditional medicine plants. It is widely distributed in the central and southern regions of China, and has been used for medicinal purposes since ancient times. It is cylindrical and has a length of 15 cm to 50 cm, a diameter of 0.15 cm to 0.3 cm, is 1 cm to 4 cm between nodes, yellow on the outside, glossy on the lower part, and has vertical wrinkles. On nodes, there are influorescence marks and remaining sheath leaves, and the entrance of sheath leaves is open. The Dendrobium candidum of the present invention can be used by purchasing those that are commercially available, or can be used by collecting those in the nature or those that are cultivated.

In one embodiment of the present invention, whole plant extracts of Dendrobium candidum, Dendrobium nubile, Dendrobium loddigessii, Dendrobium fimbriatum, and Dendrobium chrysanthum were each prepared, and wrinkle improvement effect, skin brightening effect, skin trouble improvement effect, and skin moisturizing effect of the prepared extracts were compared, respectively. As a result, it was confirmed that compared to other plants belonging to the Dendrobii Claulis class, the skin improvement effect was the most excellent when Dendrobium candidum was used (Tables 1 to 4).

In the present invention, Dendrobium candidum can be used by specifically selecting a flower part among plant parts.

In one embodiment of the present invention, the Dendrobium candidum whole plant extract, root extract, stem extract, leaf extract, and flower extract were each prepared, and wrinkle improvement effect, skin brightening effect, skin trouble improvement effect, and skin moisturizing effect of the prepared extracts were compared, respectively. Through the results of each experiment, it was confirmed that the skin improvement effect was the most excellent when using the extract derived from the flower part of Dendrobium candidum compared to using other parts (Tables 1 to 4). Therefore, the present invention is characterized in using the flower part extract among Dendrobium candidum.

As used herein, the term “extract” includes an extract itself and an extract of all formulations that can be formed using the extract such as a liquid extract obtained by the extraction process of Dendrobium candidum, a diluent or concentrate of the liquid extract, a dried product obtained by drying the liquid extract, a crude or purified product of the liquid extract, or a mixture thereof, etc.

The extract of the present invention can be extracted from natural, hybrid, or mutant plants of Dendrobium candidum, and can also be extracted from plant tissue culture.

In the extraction of the Dendrobium candidum of the present invention, a method of extracting the extract is not particularly limited, and it may be extracted according to a method commonly used in the art. Non-limiting examples of the extraction method may include hot water extraction, ultrasonic extraction, filtration, reflux extraction, etc., and these may be carried out alone or in a combination of two or more methods.

The type of extraction solvent used to extract the Dendrobium candidum in the present invention is not particularly limited, and any solvent known in the art may be used. Non-limiting examples of the extraction solvent include water (or distilled water); C1 to C4 lower alcohols such as methanol, ethanol, propyl alcohol, butyl alcohol, etc.; polyhydric alcohols such as glycerin, butylene glycol, propylene glycol, etc.; and hydrocarbon solvents such as methyl acetate, ethyl acetate, acetone, benzene, hexane, diethyl ether, and dichloromethane; or a mixture thereof, and specifically, water (or distilled water), lower alcohol, 1,3-butylene glycol, and ethyl acetate may be used alone, or two or more types thereof may be mixed, and more specifically, water (or distilled water) can be used.

The solvent extract may be prepared by extracting Dendrobium candidum one or more times by using the solvent, and a dry extract obtained by freeze-drying or spray-drying the solvent extract after distillation under reduced pressure may be prepared.

In one embodiment of the present invention, water (or distilled water) was used to prepare an extract of Dendrobium candidum (Preparation Examples 1 to 9).

The present invention specifically provides a cosmetic composition for improving wrinkles, which comprises a Dendrobium candidum flower extract as an active ingredient.

As used herein, the term “wrinkle improvement” means inhibiting or suppressing the formation of wrinkles on the skin, or alleviating wrinkles already generated.

In one embodiment of the present invention, as a result of measuring the type 1 collagen synthesis rate at the cellular level by adding the Dendrobium candidum flower extract to the culture medium of human-derived fibroblasts, the collagen synthesis rate was the most excellent compared to other types of Dendrobii Caulis and other part extracts, and it was confirmed that there was more than equivalent collagen synthesis effect than the positive control group (TGF-β) (Table 1). The Dendrobium candidum flower extract of the present invention can be effectively used for the use of wrinkle improvement, by promoting collagen synthesis.

The present invention specifically provides a cosmetic composition for skin brightening comprising a Dendrobium candidum flower extract as an active ingredient.

As used herein, the term “skin brightening” refers to not only brightening skin tone by inhibiting the synthesis of melanin pigments, but also improving skin hyperpigmentation such as blemishes and freckles caused by ultraviolet rays, hormones, or heredity.

In one embodiment of the present invention, as a result of measuring the total amount of melanin by adding the Dendrobium candidum flower extract to a mouse melanoma cell (B-16) culture solution, the inhibition rate of melanin formation was shown to be most excellent compared to other types of Dendrobii Caulis and extracts of other parts of Dendrobium candidum, and it was confirmed that there was an inhibitory effect of melanin formation that was equivalent or more of the positive control group (Albutin) (Table 2). The Dendrobium candidum flower extract of the present invention can be effectively used in the use of skin brightening because it inhibits melanin formation.

The present invention specifically provides a cosmetic composition for skin trouble improvement, which comprises a Dendrobium candidum flower extract as an active ingredient.

As used herein, the term “skin trouble” refers to skin diseases that can be caused by inflammation that occur due to excessive formation of nitrogen monoxide in macrophages. The skin trouble includes all those that are skin diseases relates to inflammation regardless of their type, and non-limiting examples of the skin diseases related to inflammation are erythematous diseases triggered by atopic dermatitis, psoriasis, radiation, chemicals, burns, etc., acid burns, bullous skin disease, lichenoid-type diseases, itching due to allergies, seborrheic eczema, rosacea, pemphigus vulgaris, erythema exsudativum multiforme, erythema nodosum, balanitis, vulvitis, inflammatory hair loss such as alopecia areata, cutaneous T-cell lymphoma, etc., and specifically include skin rashes, acne, pimples, red nose, etc., but are not limited thereto. In addition, as used herein, the term “trouble improvement” refers to suppressing or inhibiting the formation of trouble on skin, or alleviating trouble that has already formed.

As used herein, the term “anti-inflammation” refers to suppressing inflammation, and the inflammation is one type of defense responses of biological tissues against a certain stimulus, and refers to a complex lesion involving three types of tissue degeneration, circulatory disorder and exudation, and tissue proliferation. More specifically, inflammation is part of innate immunity, and as in other animals, human innate immunity recognizes patterns of cell surfaces that are specific to pathogens. Phagocytes recognize cells with such surfaces as non-self and attach pathogens. If a pathogen breaks through the body's physical barrier, an inflammatory reaction occurs. Inflammatory reactions are nonspecific defense responses that create a hostile environment against invading microbes. In inflammatory reactions, when wounds are made or external infectious agents enter the body, leukocytes responsible for the initial immune response rush to express cytokines. Therefore, the expression level of cytokines in cells is an indicator of inflammatory response activation. The anti-inflammation of the present invention may be to suppress the inflammation developed in the skin.

In one embodiment of the present invention, as a result of measuring the nitric oxide (NO) inhibitory effect of the Dendrobium candidum flower extract, inhibition rates of NO production were the most excellent compared to other types of Dendrobii Caulis and extracts of other parts of Dendrobium candidum, and it was confirmed that there was inhibitory effect of NO production that was equivalent or more compared to a positive control group (L-NMMA) (Table 3). The Dendrobium candidum flower extract of the present invention can be effectively used for the use of skin trouble improvement, by inhibiting NO production.

The present invention specifically provides a cosmetic composition for skin moisturizing, which comprises a Dendrobium candidum flower extract as an active ingredient.

As used herein, the term “skin moisturizing” means to increase moisture on the skin and keep it moist. Skin moisturizing effect can help to improve wrinkles of the skin and increase elasticity.

In one embodiment of the present invention, as a result of measuring the gene expression effect related to moisturizing of the Dendrobium candidum flower extract, it was confirmed that the promotion of moisturizing gene expression was the most improved compared to other types of Dendrobii Caulis and extracts of other parts of Dendrobium candidum (Table 4). The Dendrobium candidum flower extract of the present invention can be effectively used for the use of skin moisturizing, by promoting the moisturizing gene expression.

In summary, it was confirmed that Dendrobium candidum of the present invention has the most excellent wrinkle improvement effect, skin brightening effect, skin trouble improvement effect, and skin moisturizing effect compared to other types of Dendrobii Caulis.

In addition, it was confirmed that, among the plant parts of Dendrobium candidum, especially when using the flower part, the wrinkle improvement effect, skin brightening effect, skin trouble improvement effect, and skin moisturizing effect were the most excellent compared to using other parts.

The cosmetic composition according to the present invention can be prepared as a dosage form selected from the group consisting of a solution, external ointment, cream, foam, nourishing lotion, softening lotion, peck, softening water, milky lotion, make-up base, essence, soap, liquid detergent, bath, sunscreen cream, sun oil, suspension, emulsion, paste, gel, lotion, powder, soap, surfactant-containing cleansing, oil, powder foundation, emulsion foundation, wax foundation, patch, and spray, but is not limited thereto.

In addition, the cosmetic composition of the present invention may further include one or more cosmetically acceptable carriers formulated in general skin cosmetics, and as conventional components, for example, oil, water, surfactants, moisturizers, lower alcohols, thickeners, chelating agents, pigments, preservatives, fragrance, etc. can be suitably mixed, but is not limited thereto.

The cosmetically acceptable carrier included in the cosmetic composition of the present invention varies depending on the dosage form.

When the dosage form of the present invention is an ointment, paste, cream, or gel, the carrier component may be animal oil, vegetable oil, wax, paraffin, starch, tracant, cellulose derivative, polyethylene glycol, silicone, bentonite, silica, talc, zinc oxide, or a mixture thereof may be used.

When the dosage form of the present invention is a powder or spray, as a carrier component, lactose, talc, silica, aluminum hydroxide, calcium silicate, polyamide powder, or a mixture thereof may be used, and particularly in the case of spray, a propellant such as additionally chlorofluorohydrocarbon, propane/butane, or dimethyl ether may be additionally included.

When the dosage form of the present invention is a solution or emulsion, as a carrier component, a solvent, solubilizer, or emulsifier is used, and for example, water, ethanol, isopropanol, ethyl carbonate, ethyl acetate, benzyl alcohol, benzyl benzoate, propylene glycol, and 1,3-butylglycol oil may be used, and in particular, cottonseed oil, peanut oil, corn seed oil, olive oil, castor oil and sesame oil, glycerol alipathic ester, polyethylene glycol, or fatty acid ester of sorbitan may be used.

When the dosage form of the present invention is a suspension, as a carrier component, liquid diluents such as water, ethanol, or propylene glycol, suspending agents such as ethoxylated isostearyl alcohol, polyoxyethylene sorbitol ester, and polyoxyethylene sorbitan ester, microcrystalline cellulose, aluminum methahydroxy, bentonite, agar, or tracant, etc. may be used.

When the dosage form is soap, as a carrier component, alkali metal salts of fatty acids, fatty acid hemiester salts, fatty acid protein hydrolyzates, isethionates, lanolin derivatives, alipathic alcohols, vegetable oils, glycerol, sugars, etc. may be used.

In another aspect, the present invention provides a quasi-drug composition comprising a Dendrobium candidum flower extract as an active ingredient.

Specifically, the present invention provides a quasi-drug composition for wrinkle improvement, skin brightening, skin trouble improvement, or skin moisturizing, which comprises a Dendrobium candidum flower extract as an active ingredient.

The Dendrobium candidum flower extract, wrinkle improvement, skin brightening, skin trouble improvement, and skin moisturizing of the present invention are as described above.

The quasi-drug composition of the present invention may further include a pharmaceutically acceptable carrier, excipient, or diluent, if necessary, in addition to the above components. The pharmaceutically acceptable carrier, excipient, or diluent is not limited as long as it does not hinder the effect of the present invention, and for example, a filler, extender, binder, wetting agent, disintegrant, surfactant, lubricant, sweetener, fragrance, preservative, etc. may be included.

Representative examples of the pharmaceutically acceptable carrier, excipient, or diluent of the present invention may be lactose, dextrose, sucrose, sorbitol, mannitol, xylitol, maltitol, starch, gelatin, glycerin, acacia rubber, alginate, calcium phosphate, calcium carbonate, calcium silicate, cellulose, methyl cellulose, microcrystalline cellulose, polyvinyl pyrrolydone, water, methylhydroxybenzoate, propylhydroxybenzoate, talc, magnesium stearate, mineral oil, propylene glycol, polyethylene glycol, vegetable oil, injectable ester, witepsol, macrogol, tween 61, cacao butter, Lowry butter, etc.

In addition, when using the Dendrobium candidum flower extract of the present invention as a quasi-drug, it may further contain one or more active ingredients exhibiting the same or similar functions. For example, it may include already known wrinkle improvement, skin brightening, skin trouble improvement, and skin moisturizing ingredients. When additional wrinkle improvement, skin brightening, skin trouble improvement, and skin moisturizing ingredients are included, the wrinkle improvement, skin brightening, skin trouble improvement, and skin moisturizing effect of the composition of the present invention may be further increased. When the ingredients are added, skin safety, easy of formulation, and stability of the active ingredients may be considered for complex use.

The quasi-drug composition is a skin elasticity, wrinkle improvement, or moisturizing ingredient known in the art, and may further include one or two or more components selected from the group consisting of retinoic acid, TGF, protein from animal placenta, betulinic acid and chlorella extracts; as anti-inflammatory ingredients known in the art, non-steroidal fluphenamic acid, ibuprofen, benzidamine, indomethacin, prednisolone, dexamethasone, allantoin, azene, and hydrocortisone; and derivatives thereof and various plant extracts. The additional ingredients may be included in 0.0001% to 10% by weight relative to the total weight of the composition, and the content range may be adjusted according to requirements such as skin safety, easy in formulating the Dendrobium candidum flower extract of the present invention, etc.

The quasi-drug composition of the present invention may be exemplified by disinfecting a detergent, a shower foam, an ointment, a wet tissue, a coating agent, etc., but is not limited thereto, and formulation methods, dosages, methods of use, constituting components, etc. of the quasi-drug can be appropriately selected from conventional techniques known in the art.

In another aspect, the present invention provides a food composition comprising a Dendrobium candidum flower extract as an active ingredient.

Specifically, the present invention provides a food composition for wrinkle improvement, skin brightening, skin trouble improvement, or skin moisturizing, which comprises a Dendrobium candidum flower extract as an active ingredient.

The Dendrobium candidum flower extract, wrinkle improvement, skin brightening, skin trouble improvement, and skin moisturizing of the present invention are as described above.

The Dendrobium candidum flower extract included in the food composition of the present invention may be included in the form of animals and plants including a Dendrobium candidum flower extract, an extract thereof, a fraction thereof, or a processed product thereof. In addition, the composition may include a food supplement that is cytologically acceptable in addition to the active ingredient.

As used herein, the term “food supplementary additive” means a component that can be added to food, and as it is added to prepare health functional food in each dosage form, those skilled in the art can appropriately select and use it. Examples of food supplementary additives include a flavoring agent such as various nutrients, vitamins, minerals (electrolytes), synthetic and natural flavors, etc., colorants and fillers, pectic acid and salts thereof, alginic acid and salts thereof, organic acids, protective colloidal thickeners, pH regulators, stabilizers, preservatives, glycerin, alcohols, carbonation agents used in carbonated beverages, etc., but the type of food supplementary additive of the present invention is limited by the examples above.

The food composition of the present invention may include a health functional food. As used herein, “health functional food” refers to a food prepared and processed in the form of tablets, capsules, powders, granules, liquids and pills, etc. using raw materials or ingredients having useful functions for the human body. In particular, “functional” means obtaining a useful effect for health purposes, such as nutrient control, physiological action, etc. on the structure and function of the human body. The health functional food of the present invention can be prepared by a method commonly known in the art, and upon preparation, it can be prepared by adding raw materials and ingredients commonly added in the art. In addition, the dosage form of the health functional food can also be prepared without limitation as long as the dosage form is recognized as a health functional food. The food composition of the present invention can be prepared in various forms of dosage forms, and unlike the general medicine, it has an advantage that there is no side effect that may occur upon long-term administration of the drug because it has food as a raw material, and as it has excellent portability, the health functional food of the present invention can be taken as an adjuvant for increasing wrinkle improvement, skin brightening, skin trouble improvement, and skin moisturizing effect.

There is no limitation on the form that the health functional food of the present invention can take, and may include all foods in the conventional meaning, and can be mixed with terms known in the art such as functional foods, etc. Moreover, the health functional food of the present invention may be prepared by mixing known additives with other appropriate supplementary ingredients that may be included in the food according to the choice of those skilled in the art. Examples of foods that it can be added to include meat, sausages, bread, chocolate, candy, snacks, confectionery, pizza, ramen, other noodles, gums, diary products including ice cream, various soups, beverages, teas, drinks, alcoholic beverages, vitamin complexes, etc., and it can be prepared by adding the compound represented by Chemical Formula 1 according to the present invention in a concentrate, tea, jelly and juice as a main component. In addition, it includes foods used as feed for animals.

In addition, the Dendrobium candidum flower extract of the present invention can be used for wrinkle improvement, skin brightening, skin trouble improvement, or skin moisturizing method, comprising a step of applying to the skin of a subject. The subject includes, without limitation, mammals including rats, livestock, humans, etc.

Further, the present invention provides a wrinkle improvement method, a skin brightening method, a skin trouble improvement method, or a skin moisturizing method comprising a step of applying a Dendrobium candidum flower extract to a subject's skin.

The Dendrobium candidum flower extract, wrinkle improvement, skin brightening, skin trouble improvement, and skin moisturizing of the present invention are as described above.

The present invention provides an anti-wrinkle use, skin brightening use, skin trouble improvement use, or skin moisturizing use of the cosmetic composition comprising a Dendrobium candidum flower extract.

In addition, the present invention provides an anti-wrinkle use, skin brightening use, skin trouble improvement use, or skin moisturizing use of the quasi-drug composition comprising a Dendrobium candidum flower extract.

In addition, the present invention provides an anti-wrinkle use, skin brightening use, skin trouble improvement use, or skin moisturizing use of the food composition comprising a Dendrobium candidum flower extract.

The Dendrobium candidum flower extract, wrinkle improvement, skin brightening, skin trouble improvement, and skin moisturizing are as described above.

The present invention provides a method for preparing a cosmetic composition for wrinkle improvement, a cosmetic composition for skin brightening, a cosmetic composition for skin trouble improvement, or a cosmetic composition for skin moisturizing, comprising a Dendrobium candidum flower extract as an active ingredient.

In addition, the present invention provides a method for preparing a quasi-drug composition for wrinkle improvement, a quasi-drug composition for skin brightening, a quasi-drug composition for skin trouble improvement, or a quasi-drug composition for skin moisturizing, comprising a Dendrobium candidum flower extract as an active ingredient.

In addition, the present invention provides a method for preparing a food composition for wrinkle improvement, a food composition for skin brightening, a food composition for skin trouble improvement, or a food composition for skin moisturizing, comprising a Dendrobium candidum flower extract as an active ingredient.

The Dendrobium candidum flower extract, wrinkle improvement, skin brightening, skin trouble improvement, and skin moisturizing are as described above.

### DETAILED DESCRIPTION

Hereinafter, the present invention will be described in more detail through examples. These examples are intended to illustrate the present invention more specifically, but the scope of the present invention is not limited to these examples.

### Preparation Examples 1 to 5: Preparation of Whole Plant Extract of 5 Types of Dendrobii Claudis

### <Preparation Example 1> Preparation of Whole Plant Extract of Dendrobium candidum

After drying the whole plant (flowers, leaves, stems, and roots) of Dendrobium candidum, 200 g of distilled water was added to 100 g of dry weight and extracted for 3 hours at 80° C. The obtained extract was filtered through a filter having a pore size of 0.2 μm to prepare a Dendrobium candidum whole plant extract.

### <Preparation Example 2> Preparation of Whole Plant Extract of Dendrobium nubile

Except that Dendrobium candidum was replaced with Dendrobium nobile, it was prepared in the same manner as in Preparation Example 1.

### <Preparation Example 3> Preparation of Whole Plant Extract of Dendrobium loddigesii

Except that Dendrobium candidum was replaced with Dendrobium loddigesii, it was prepared in the same manner as in Preparation Example 1.

### <Preparation Example 4> Preparation of Whole Plant Extract of Dendrobium fimbriatum

Except that Dendrobium candidum was replaced with Dendrobium fimbriatum, it was prepared in the same manner as in Preparation Example 1.

### <Preparation Example 5> Preparation of Whole Plant Extract of Dendrobium chrysanthum

Except that Dendrobium candidum was replaced with Dendrobium chrysanthum, it was prepared in the same manner as in Preparation Example 1.

### Preparation Examples 6 to 9: Preparation of Extract for Each Part of Dendrobium candidum

### <Preparation Example 6> Preparation of Root Extract of Dendrobium candidum

Except that the whole plant of Dendrobium candidum was replaced with the root of Dendrobium candidum, it was prepared in the same manner as in Preparation Example 1.

### <Preparation Example 7> Preparation of Stem Extract of Dendrobium candidum

Except that the whole plant of Dendrobium candidum was replaced with the stem of Dendrobium candidum, it was prepared in the same manner as in Preparation Example 1.

### <Preparation Example 8> Preparation of Leaf Extract of Dendrobium candidum

Except that the whole plant of Dendrobium candidum was replaced with the leaf of Dendrobium candidum, it was prepared in the same manner as in Preparation Example 1.

### <Preparation Example 9> Preparation of Flower Extract of Dendrobium candidum

Except that the whole plant of Dendrobium candidum was replaced with the flower of Dendrobium candidum, it was prepared in the same manner as in Preparation Example 1.

### Experimental Example 1: Analysis of Synthesis Promotion Effect of Type 1 Collagen in Human-Derived Fibroblast

In order to confirm the wrinkle improvement effect through promoting the synthesis of type 1 collagen of each extract prepared in Preparation Examples 1 to 9, collagen synthesis rate was measured by adding the samples obtained in Preparation Examples 1 to 9 to the culture solution of human-derived fibroblasts. The synthesis of collagen was quantified using a PICP EIA kit (Procollagen Type I C-Peptide Enzyme ImmunoAssay KIT). After adding the sample to a culture medium of fibroblasts (DMEM medium) at a final concentration of 0.1% and culturing for 48 hours, cultures were taken and the degree of collagen synthesis of type 1 collagen at each concentration was measured at 450 nm using a spectrophotometer with PICP EIA kit.

For comparison of the effects, the degree of collagen synthesis was measured in the same manner with respect to a culture medium (negative control group) of untreated fibroblasts and the sample (positive control) added with TGF-β to a final concentration of 10 ng/mL.

Collagen synthesis rate (%) was calculated as the ratio of the collagen production amount relative to the negative control, and the results are shown in Table 1 below.

$$Collagen synthesis rate ( \% ) = \frac{\begin{matrix}absorbance of sample treated qroup - \\ absorbance of untreated qroup\end{matrix}}{absorbance of untreated group} \times 100$$

TABLE 1

| Sample | Absorbance (abs) | Collagen synthesis rate (%) |
|---|---|---|
| Negative control group | 1.585 | 0 |
| TGF-β (10 ng/ml) | 1.826 | 15.21 |
| Preparation Example 1 | 1.763 | 11.23 |
| (0.1%) | | |
| Preparation Example 2 | 1.653 | 4.29 |
| (0.1%) | | |
| Preparation Example 3 | 1.692 | 6.75 |
| (0.1%) | | |
| Preparation Example 4 | 1.617 | 2.02 |
| (0.1%) | | |
| Preparation Example 5 | 1.628 | 2.71 |
| (0.1%) | | |
| Preparation Example 6 | 1.601 | 1.01 |
| (0.1%) | | |
| Preparation Example 7 | 1.723 | 8.71 |
| (0.1%) | | |
| Preparation Example 8 | 1.701 | 7.32 |
| (0.1%) | | |
| Preparation Example 9 | 1.892 | 19.37 |
| (0.1%) | | |

(Number of repetitions = 4)

As shown in Table 1 above, when treated with a Dendrobium candidum extract (Preparation Example 1), it was confirmed that the collagen synthesis rate was the most excellent compared to cases of being treated with each of a Dendrobium nobile, Dendrobium loddigesii, Dendrobium fimbriatum, or Dendrobium chrysanthum extract (Preparation Examples 2 to 5).

In addition, when treated with an extract derived from the flower of Dendrobium candidum, it was confirmed that the collagen synthesis rate was the most excellent compared to being treated with the root, stem, or leaf extract of Dendrobium candidum (Preparation Examples 6 to 8), and this was more excellent than the treatment of the whole plant extract (Preparation Example 1) of Dendrobium candidum.

Additionally, the Dendrobium candidum flower extract (Preparation Example 9) showed a collagen synthesis effect that is equal to or better than that of applying TGF-β (positive control group), which is generally known to induce collagen synthesis.

Accordingly, it was known that the Dendrobium candidum flower extract effectively promoted collagen synthesis compared to other types of Dendrobii Claulis and other part extracts of Dendrobium candidum, and it can be usefully applied for the use of skin wrinkle improvement using the same.

### Experimental Example 2: Analysis of Inhibitory Effect of Melanin Production at Cellular Level

In order to confirm the brightening effect through the inhibition of melanin production of each extract prepared in Preparation Examples 1 to 9, the samples obtained in Preparation Examples 1 to 9 were added to a culture medium of mouse melanoma cells (B-16 mouse melanoma cell) to measure the total amount of melanin (Lotan R. et al., Cancer Res. 40:3345-3350, 1980). Prior to measuring the total amount of melanin, brightening evaluation was performed at non-toxic concentrations by first evaluating the toxicity of mouse melanoma cells.

The samples of Preparation Examples 1 to 9 were each added to a medium to a final concentration of 0.1% and B-16 melanoma cells were cultured for 3 days, by adding to a sample-untreated medium (negative control group), and to a medium of albutin to 100 ppm (positive control group), respectively.

Thereafter, the cells were trypsin-treated, detached from the culture vessel, and centrifuged, and melanin was extracted. The detached cells were added with 1 mL of sodium hydroxide solution (1N concentration), boiled for 10 minutes to dissolve the melanin, and the absorbance was measured at 405 nm using a spectrophotometer to measure the amount of melanin produced.

The amount of melanin was measured by the absorbance of the unit cell count (1×10⁶ cells), and the melanin production inhibition rate (%) was calculated as the total amount of melanin relative to the negative control group, and the results are shown in Table 2 below.

$$Melanin production inhibition rate ( \% ) = 100 - \frac{Absorbance of sample treated group}{Absorbance of negative control group} \times 100$$

TABLE 2

| | | Melanin production |
|---|---|---|
| | | inhibition |
| Sample | Absorbance (abs) | rate (%) |
| Negative control group | 0.652 | 0 |
| Albutin (100 ppm) | 0.436 | 33.13 |
| Preparation Example 1 (0.1%) | 0.523 | 19.79 |
| Preparation Example 2 (0.1%) | 0.615 | 5.67 |
| Preparation Example 3 (0.1%) | 0.623 | 4.45 |
| Preparation Example 4 (0.1%) | 0.596 | 8.59 |
| Preparation Example 5 (0.1%) | 0.633 | 2.91 |
| Preparation Example 6 (0.1%) | 0.614 | 5.83 |
| Preparation Example 7 (0.1%) | 0.571 | 12.42 |
| Preparation Example 8 (0.1%) | 0.581 | 10.89 |
| Preparation Example 9 (0.1%) | 0.413 | 36.66 |

(Number of repetitions = 4)

As shown in Table 2 above, when treated with a Dendrobium candidum extract (Preparation Example 1), it was confirmed that the melanin production inhibition rate was the most excellent compared to cases of being treated with each of a Dendrobium nobile, Dendrobium loddigesii, Dendrobium fimbriatum, or Dendrobium chrysanthum extract (Preparation Examples 2 to 5).

In addition, when treated with an extract derived from the flower of Dendrobium candidum (Preparation Example 9), it was confirmed that the melanin production inhibition rate was the most excellent compared to being treated with the root, stem, or leaf extract of Dendrobium candidum (Preparation Examples 6 to 8), and this was more excellent than the treatment of the whole plant extract (Preparation Example 1) of Dendrobium candidum.

Additionally, the Dendrobium candidum flower extract (Preparation Example 9) showed a melanin inhibitory effect that is equal to or better than that of albutin treatment (positive control group), which is generally known to inhibit melanin production.

Accordingly, it was known that the Dendrobium candidum flower extract effectively inhibited melanin production compared to other types of Dendrobii Claulis and other part extracts of Dendrobium candidum, and it can be usefully applied for the use of skin brightening using the same.

### Experimental Example 3: Analysis of Inhibitory Effect of Nitric Oxide (NO) Production

In order to confirm the anti-inflammatory effect and skin trouble improvement effect of each extract prepared in Preparation Examples 1 to 9, NO production inhibitory effect was measured by the GRIESS method using RAW264.7 cell line (ATCC number: CRL-2278).

Specifically, RAW264.7 cells, which are macrophages of mice, were subcultured several times, and cultured for 24 hours after being placed in a 24-well plate such that 3×10⁵ cells were placed in one well. After exchanging with a cell medium containing the sample at a final concentration of 0.1%, an untreated medium was used as a negative group and for a positive control group, 20 ppm of L-NG-monomethylarginine (L-NMMA) was used, which is a NO-production inhibitor. 1 ppm of lipopolysaccharide was treated as a stimulus source and cultured for 24 hours. The supernatant was transferred to a 96-well plate by 100 μL, and the GRIESS solution was added by 100 μL to react at room temperature for 10 minutes, and the NO inhibitory effect was determined by measuring the absorbance at 540 nm.

The NO production inhibitory rate (%) was calculated as a relative ratio of the amount of reduced NO compared to the LPS-only treatment group, and the results are shown in Table 3 below.

$$NO production inhibitory effect = \frac{\begin{matrix}Absorbance of sample treated group - \\ absorbance of untreated group\end{matrix}}{\begin{matrix}Absorbance of LPS treated group - \\ absorbance of untreated group\end{matrix}} \times 100$$

TABLE 3

| | | NO production |
|---|---|---|
| Sample | Absorbance (abs) | inhibitory rate (%) |
| Negative control group | 0.043 | — |
| LPS treated group | 0.232 | 0 |
| L-NMMA (20 ppm) | 0.134 | 51.85 |
| Preparation Example 1 (0.1%) | 0.173 | 31.22 |
| Preparation Example 2 (0.1%) | 0.213 | 10.05 |
| Preparation Example 3 (0.1%) | 0.224 | 4.23 |
| Preparation Example 4 (0.1%) | 0.199 | 17.46 |
| Preparation Example 5 (0.1%) | 0.217 | 7.94 |
| Preparation Example 6 (0.1%) | 0.219 | 6.88 |
| Preparation Example 7(0.1%) | 0.181 | 26.98 |
| Preparation Example 8 (0.1%) | 0.195 | 19.58 |
| Preparation Example 9 (0.1%) | 0.131 | 53.44 |

(Number of repetitions = 4)

As shown in Table 3 above, when treated with a Dendrobium candidum extract (Preparation Example 1), it was confirmed that the NO production inhibition rate was the most excellent compared to cases of being treated with each of a Dendrobium nobile, Dendrobium loddigesii, Dendrobium fimbriatum, or Dendrobium chrysanthum extract (Preparation Examples 2 to 5).

In addition, when treated with an extract derived from the flower of Dendrobium candidum (Preparation Example 9), it was confirmed that the NO production inhibition rate was the most excellent compared to being treated with the root, stem, or leaf extract of Dendrobium candidum (Preparation Examples 6 to 8), and this was more excellent than the treatment of the whole plant extract (Preparation Example 1) of Dendrobium candidum.

Moreover, the Dendrobium candidum flower extract (Preparation Example 9) showed a NO production inhibitory effect that is equal to or better than that of L-NMMA treatment, which is a representative anti-inflammatory ingredient.

Accordingly, it was known that the Dendrobium candidum flower extract effectively inhibited NO production compared to other types of Dendrobii Claulis and other part extracts of Dendrobium candidum and thus had excellent anti-inflammatory effect, and it can be usefully applied for the use of skin trouble improvement using the same.

### Experimental Example 4: Analysis of Gene Expression Effect Related to Moisturizing

In order to confirm the skin moisturizing effect of each extract prepared in Preparation Examples 1 to 9, gene expression related to moisturizing was measured using the cells cultured as below.

Human keratinocytes (HaCaT) were in a 6-hole cell culture plate 2×10⁵ each, and then cultured using Dulbecco's Modified Eagle's medium (DMEM, Gibco, Co.) including fetal bovine serum (FBS) and antibiotics for 24 hours.

After culturing the cells, it was replaced with DMEM, High Glucose, no Glutamine, no calcium (Gibco, USA), and then the samples obtained in Preparation Examples 1 to 9 were treated at a concentration of 0.1% for 24 hours. After the cells were recovered and washed with phosphate buffer solution (PBS), total RNA was extracted using an RNA extraction kit (RNeasy mini kit, Qiagen, Germany).

2.5 μg of RNA was quantified and reverse transcription was performed using cDNA synthesis kit (PhileKorea, Korea). Reverse transcription was performed using a Veriti 96 well Thermal Cycler (Applied Biosystems, USA), and the synthesized cDNA was used by 100 ng per reaction, and TaqMan® Universal Master Mix II was used. Quantitative real time PCR (qRT-). PCR) was performed with AQP3, FLG, HAS2, and HAS3 primers (TaqMan® Gene Expression Assays, Thermo Fisher, USA) that were run on a StepOnePlus® Real-time PCR System (Applied Biosystems, USA).

The experimental results obtained by qRT-PCT were shown by calculating with the ΔΔCT method (Livak K J, Schmittgen T D: Analysis of relative gene expression data using real-time quantitative PCR and the 2(-Delta Delta C(T)) Method, Methods, 25, 402-408(2001)) based on housekeeping gene and glyceraldehyde-3-phosphate dehydrogenase (GAPDH). To compare effects, moisturizing gene expression was measured in the same manner for a negative control group without treating samples and a positive control group treated with EGF 10 ng/ml and Ca²⁺3 mM, respectively.

The results of quantifying the mRNA expression level of the treated experimental group based on the mRNA expression level of the negative control group, which was not treated, as 1.00 were shown in Table 4.

TABLE 4

| | | Hyaluronic acid | Hyaluronic acid |
|---|---|---|---|
| | Aquaporin 3 | synthase 2 | synthase 3 |
| Sample | (fold) | (fold) | (fold) |
| Negative control | 1.00 | 1.00 | 1.00 |
| group | | | |
| EGF (10 ng/ml) | 3.27 | — | — |
| Ca²⁺ (3 mM) | — | 2.01 | 3.60 |
| Preparation Example | 1.51 | 1.73 | 1.82 |
| 1 (0.1%) | | | |
| Preparation Example | 112 | 1.23 | 1.18 |
| 2 (0.1%) | | | |
| Preparation Example | 1.09 | 1.11 | 1.04 |
| 3 (0.1%) | | | |
| Preparation Example | 1.15 | 1.13 | 1.09 |
| 4 (0.1%) | | | |
| Preparation Example | 1.21 | 1.28 | 1.26 |
| 5 (0.1%) | | | |
| Preparation Example | 1.05 | 1.09 | 1.10 |
| 6 (0.1%) | | | |
| Preparation Example | 1.51 | 1.64 | 1.69 |
| 7 (0.1%) | | | |
| Preparation Example | 1.42 | 1.53 | 1.56 |
| 8 (0.1%) | | | |
| Preparation Example | 2.75 | 2.93 | 2.96 |
| 9 (0.1%) | | | |

(Number of repetitions = 4)

As shown in Table 4 above, when treated with a Dendrobium candidum extract (Preparation Example 1), it was confirmed that the expression of all of genes related to skin moisturizing used in the experiment was the most excellent compared to cases of being treated with each of a Dendrobium nobile, Dendrobium loddigesii, Dendrobium fimbriatum, or Dendrobium chrysanthum extract (Preparation Examples 2 to 5).

In addition, when treated with an extract derived from the flower of Dendrobium candidum (Preparation Example 9), it was confirmed that the expression of the genes related to skin moisturizing was the most excellent compared to being treated with the root, stem, or leaf extract of Dendrobium candidum (Preparation Examples 6 to 8), and this was more excellent than the treatment of the whole plant extract (Preparation Example 1) of Dendrobium candidum.

Accordingly, it was known that the Dendrobium candidum flower extract showed excellent skin moisturizing effect compared to other types of Dendrobii Claulis and other part extracts of Dendrobium candidum, and it can be usefully applied for the use of skin moisturizing using the same.

Through the results of Experimental Examples 1 to 4 above, the Dendrobium candidum extract according to the present invention has more excellent skin improvement effect (wrinkle improvement, skin brightening, skin trouble improvement, and skin moisturizing) compared to Dendrobium nobile, Dendrobium loddigesii, Dendrobium fimbriatum, and Dendrobium chrysanthum extracts, and it was confirmed that the effect was the most excellent when using an extract obtained from the flower part in Dendrobium candidum.

Therefore, the Dendrobium candidum flower extract can be effectively used in compositions for wrinkle improvement, skin brightening, skin trouble improvement, or skin moisturizing.

From the above description, those skilled in the art will appreciate that the present invention can be implemented in other specific forms without changing the technical spirit or essential features. In this regard, the embodiments described above are to be understood in all respects as illustrative and not restrictive. The scope of the present invention should be construed that all changes or modifications derived from the meaning and scope of the following claims and equivalent concepts rather than the detailed description are included in the scope of the present invention.

1. A method for improving skin condition, comprising:
applying a cosmetic composition comprising a Dendrobium candidum flower extract to the skin of a subject.

2. The method of claim 1, wherein the improvement of skin condition is wrinkle improvement.

3. The method of claim 1, wherein the improvement of skin condition is skin brightening.

4. The method of claim 1, wherein the improvement of skin condition is skin trouble improvement.

5. The method of claim 1, wherein the improvement of skin condition is skin moisturizing.

6. The method of claim 1, wherein the cosmetic composition has collagen synthesis promoting effect, melanin production inhibitory effect, anti-inflammatory effect, or moisturizing effect.

7. A method for improving skin condition, comprising:
administrating a food composition comprising a Dendrobium candidum flower extract to a subject.

8. The method of claim 7, wherein the improvement of skin condition is wrinkle improvement.

9. The method of claim 7, wherein the improvement of skin condition is skin brightening.

10. The method of claim 7, wherein the improvement of skin condition is skin trouble improvement.

11. The method of claim 7, wherein the improvement of skin condition is skin moisturizing.

12. The method of claim 7, wherein the food composition has collagen synthesis promoting effect, melanin production inhibitory effect, anti-inflammatory effect, or moisturizing effect.
